(19) United States
(12) Patent Application Publication
(10) Pub. No.: US 2019/0126042 A1
(43) Pub. Date: May 2, 2019

(54) SYSTEM AND METHOD APPLYING ELECTRICAL SIGNAL TO TREAT PATHOGEN AND RELATED DISEASE

(71) Applicant: JUNWU ZHANG, Xi'an (CN)

(72) Inventor: JUNWU ZHANG, Xi'an (CN)

(21) Appl. No.: 16/094,883

(22) PCT Filed: Apr. 19, 2017
(86) PCT No.: PCT/CN2017/081049

§ 371 Date: Oct. 19, 2018

Related U.S. Application Data
(60) Provisional application No. 62/324,701, filed on Apr. 19, 2016.

(51) Int. Cl. A61N 1/36; A61N 1/04

(52) U.S. Cl. CPC A61N 1/36034; A61N 1/0408

(57) ABSTRACT

An apparatus is used for applying electrical field or electrical current to treat pathogens in a subject (e.g. mammal) or in any body fluid or synthetic fluid. The apparatus comprises at least one pair of conductive electrodes each of which connects to physically separated points on the body, or on a vessel containing body fluid or synthetic fluid, and at least one electrical signal generator circuitry which generates oscillatory signal of selected frequency, waveform, and voltage.

The present application claims priority from U.S. patent application 62324701 filed Apr. 19, 2016, and the entire contents of the U.S. patent application being incorporated herein by reference.

### FIELD OF THE INVENTION

This invention relates to an apparatus and method for applying electrical field or electrical current to treat pathogens and/or the related diseases.

### BACKGROUND OF THE INVENTION

It has been known from previous inventions that electrical current of certain property can be introduced in body/tissue of a subject such as a mammal to treat various diseases or illnesses caused by various pathogens.

There are two types of theories regarding the frequency of the electrical current wave to disable pathogens or treat conditions. The first theory is based on the theory that each type of pathogen such as virus, bacterium, fungus or other microorganism has distinct intrinsic frequencies related to its structure or its components such as DNA, shell, or membrane. Electrical signal at such intrinsic frequencies or its higher harmonics with proper strength can cause the pathogen to be destroyed. Most devices based on this theory use frequencies more than 50 kHz. In this theory, since each type of microorganism has different set of intrinsic frequencies, when a subject is to be treated without knowing the exact types of pathogens, the best way is to scan a range of frequencies that covers most of the known frequencies for different types of organism. In this type of treatment, there is a trade-off between coverage of pathogens and time. When the treatment needs to cover more types of pathogen at a wider range of frequency, more time is needed for such scanning. If the treatment needs to save time, the scanning has to be performed in a smaller range of frequency, or use bigger steps to scan, both of which will lead to a smaller coverage. It is known that at the correct frequency and proper electrical field strength, it takes a few tens of seconds to a few minutes to disable the pathogen, depending on the amount of the pathogen exposed.

The second theory of treating pathogens uses only fixed frequency to treat all pathogens. This was first introduced by Dr. Hulda Clark's book in 1995 “The Cure for all Diseases”, in which a method of using positive offset electrical signal of 30 kHz and 8-9V was disclosed to disable pathogens.

U.S. Pat. No. 8,781,573 by Patrick Murphy (hereafter the '573 patent), U.S. Pat. No. 5,690,692 by Janet Fleming (hereafter the '692 patent) and U.S. Pat. No. 5,925,071 by Craig Story (hereafter the '071 patent) are examples for the first theory. Hulda Clark's device can be categorized as the embodiment of the second theory.

The '573 patent disclosed an apparatus utilizing electrical signal with multiple frequencies, ranging preferably from 1 MHz to 5.4 MHz, wherein the frequency is increased in steps of 1 kHz or more. The voltage output of the electrical signal is 0.1V to 25V, more preferably less than 10V, even more preferably less than 6V. The output signal is transferred to a mammal body via a pair of conductive electrodes.

The '692 patent disclosed a precise frequency synthesizer to generate signals at 0.00004 Hz to 3 MHz as a square wave with a 50% duty cycle. The device doesn't use one frequency for different conditions. It will be set to frequencies specific to a condition. The '692 patent described hundreds of frequencies to be used to treat different conditions.

The '071 patent disclosed a device for ridding human body of pathogens with an output signal of 9V whose frequency was varied from 70 kHz to 880 kHz in 1 kHz steps. The '071 patent also disclosed a brief respite period between each cycle.

Devices based on the '573 patent, the '692 patent, and the '071 patent used different frequencies for different pathogens, which is the first theory. Such device has to have scanning on a frequency range in order to be able to deactivate pathogens, which varies from person to person, and is unknown in most cases. In addition to the scanning, many devices also provide the capability to output multiple frequencies simultaneously. For example, the device in the '573 patent employed up to 10 frequencies at the same time.

Devices established on each of the two theories were tested. It was found that scanning on a frequency range is time-consuming. The device of the '692 patent requires the exact condition or pathogen to be known, which is not feasible for people having clinical symptoms with no knowledge of the type of pathogens. The device using a fixed frequency of the second theory is more attractive. However, experiments of using different versions of Hulda Clark device at 30 kHz revealed that although these devices have some effects, the effectiveness needs to be improved.

There is a need for improved device and method for applying electrical field or electrical current to treat pathogen and related disease in a subject.

### SUMMARY OF THE INVENTION

This invention relates to an apparatus and method for applying electrical field or electrical current to treat pathogens in a subject, for example, a mammal, or in any body fluid or synthetic fluid. The apparatus comprises at least one pair of conductive electrodes each of which connects to physically separated points on a subject's body, or on a vessel containing body fluid or synthetic fluid; at least one electrical signal generator circuitry which generates oscillatory signal of selected frequency, waveform, and voltage; and one controlling circuitry which interfaces between an user and the electrical signal generator to control the voltage, waveform, electricity and the treatment period. In one embodiment, the frequency used is less than 10 kHz. The selected waveform could be any waveform, for example, bi-phasic or square waveform. In one embodiment, the absolute voltage between the electrodes is no more than 27V, for example, between 7V and 18V. In one embodiment, the treatment period is less than 10 hours, for example, between 15 minutes and 2 hours. The controlling circuitry is used by a user to control the voltage, waveform, electricity and adjust the length of one treatment period.

When the electrodes are connected to physically separated points of a vessel containing body fluid or synthetic fluid, either the pair of electrodes directly contacts the said fluids at physically separated points, or the pair of electrodes contact pair of built-in conductors which in turn contact physically separated points of the said fluid.

In contrast to previous patents, the present invention found that it is not necessary to use multiple frequencies, or scanning a range of frequencies to treat different pathogens and the related diseases. Instead, it was found that any frequency from 4 Hz to 10K kHz, which is much lower than those disclosed in the publications discussed above, can be used to disable different types of pathogens. In contrast to Dr. Hulda Clark's device featured by the fixed frequency of 30 kHz and a voltage less than 9V, the present invention is characterized by the lower frequency, higher voltage (up to 27 V, for example, 7 V to 18 V, with 7-12 V for kids and 12-18 V for adults), which can lead to be more effective and efficient therapeutic effect. In addition, this present invention found that the essentially required waveform as in Dr. Hulda Clark's device, i.e., the positively offset waveform, is not necessary for the device disclosed in the present invention to treat the pathogens and related medical conditions.

### BRIEF DESCRIPTION OF THE FIGURES

FIG. 1 shows a schematic diagram of a device with three modules.

FIG. 2 shows a schematic diagram of a device with four modules.

FIG. 3 shows a schematic diagram of a device with five modules.

FIG. 4 shows one embodiment of the present invention.

FIG. 5 shows a schematic diagram of one device having multiple pairs of electrodes to treat multiple subjects simultaneously.

FIG. 6 shows a schematic diagram of two electrodes configured on the left hand of a subject.

FIG. 7 shows a schematic diagram of two electrodes separately configured on different hands of a subject.

FIG. 8 shows a schematic diagram of two electrodes externally configured on a container containing body fluid such as blood.

### DETAILED DESCRIPTION OF THE INVENTION

The device disclosed in Dr. Hulda Clark's book utilizes a fixed frequency of 30 kHz. It should be noted that the device used positively offset signals, which means the signal is always greater than 0V. Therefore, one electrode always holds higher electrical potential than the other. Prolonged treatment with such parameters could significantly displace the ions in the subject's body, and interfere with normal biological process. To avoid such undesirable effect due to long treatment time (usually, more than 10 minutes), the present invention utilizes a much lower frequency with any waveform, for example, bi-phasic waveform which is symmetric between the two electrodes, square waveform, or any other waveform which is symmetric between the positive and negative sides, and is not positively offsetted. As a result, there is no net ion displacement in body fluid. In additional, the present invention disclosed treatment under a lower frequency (e.g. 4 Hz to 10K kHz) is more effective and efficient than under a high frequency at 30 kHz. The present invention does not require scanning in a range of frequencies. The frequency used is not pathogen-specific or disease-specific, but is fixed during the treatment.

The voltage output of Hulda Clark devices disclosed in prior publications is less than 9V. In one embodiment, the present invention found that a higher voltage output, i.e, higher than 9V, for example, 15V-18V, can provide additional benefits. Usually, a voltage higher than 27V may cause some tingling sensation of skin at the electrode contacting area. In another embodiment, a voltage less than 27V is utilized for such treatment. In another embodiment, a voltage of 18V is used to treat an adult subject without causing any uncomfortable feeling. In another embodiment, a voltage of 9V is used to treat a kid without causing any uncomfortable feeling.

Depending on the nature of the pathogen or disease, the present invention recommends using the device for between 5 minutes and 10 hours for one treatment session, for example, between 30 minutes and 2 hours. Treatment may be repeated until the disease is healed.

In one embodiment, the treatment procedure comprises applying the same frequency for more than 5 minutes. In another embodiment, the treating period is higher than 30 minutes.

In one embodiment, the scanning of frequency is not required in the present invention. Only one fixed frequency is generated and utilized to treat all diseases.

In one embodiment, the fixed frequency for special subjects, such as medicine-taker and alcohol-drinker, shall be higher than 30 Hz to avoid side effects related to change in the permeability of blood cell membrane. For alcohol drinker, the side effects can be experienced as being drunk upon drinking a small amount of alcohol, as if the alcohol's effect is magnified many folds when the subject is exposed to such treatment. The study on subjects who took medicines or drank alcohol indicates that very low frequency such as 3-8 Hz would cause side effect by magnifying the alcohol/medicine effect by many folds. As for a frequency higher than 10 Hz but less than 30 Hz, this side effect is much smaller. When a frequency higher than 30 Hz is used, no such side effect is observed. Based on prior research in DNA transfection (Biophysical Journal, Vol 58, Oct. 1990, 897-903), it can be inferred that the permeability of blood cell membrane increases when exposed to an external AC field. The highest permeability change happens at about 1 Hz. It quickly becomes less significant with the increase of frequency. The change at 30 Hz is in the order of one percent of that at 1 Hz.

In one embodiment, the alcohol effect was also tested by drinking alcohol 10 minutes after treatment with the present invention. The result indicated that no magnified alcohol effect was observed at either the very low frequency of 3 Hz or frequency of 100 Hz. This indicates the permeability change disappeared 10 minutes or sooner after completion of such treatment. In another embodiment, at a constant frequency, different voltage waveform between the pair of electrodes can be applied, such as square wave, sinusoid wave, sawtooth wave, triangle wave, bi-phasic, or superimposed on a positive offset, though positive offset is not a must. In another embodiment, the waveform can also be damped in amplitude, or intermittent, or any special waveform, even random waveform, or combination of different waveforms.

In one embodiment, a treatment period is more than 5 minutes. In another embodiment, the biphasic waveform is used, which will not permanently displace ions in body fluid or other fluid.

In one embodiment, the device comprises a power module, a waveform generator module, a waveform delivery module, which includes at least one pair of electrodes. In another embodiment, the device may also include a control module, which controls the waveform generator to synthesize the waveform, voltage, as well as timing during the whole treatment course. In another embodiment, the device may also comprise one component storing predesigned treatment program. In another embodiment, the device may also include a user interface module, which may accept user input, such as length of treatment, program of choice, and information feedback to user by means of audio output or visual display such as a LCD display.

In one embodiment, the power module may include transformer to use the household or industrial power supply, or be powered by battery. It connects to all the other modules to supply power. It may include an on/off switch.

In one embodiment, the waveform generator can include analog circuit to create analog waveform, or digital circuit such as Direct Digital Synthesis (DDS) chip synthesized waveform, or a combination of both analog and digital circuits. In another embodiment, the waveform generator receives controlling signals from the control module and provides output to the waveform delivery module.

In one embodiment, the waveform delivery module may have one or more pairs of electrodes. The electrodes can be made of any conductive material such as metal or the electrodes used in TENS or EKG system, or non-conductive material combined with conductive material or conductive fluid.

In one embodiment, the control module controls how the other modules, especially the waveform generator module, work, including how long, what waveform, what voltage the output will be. In another embodiment, the control module can be programmable or non-programmable. In another embodiment, the control module may include memory to stored information or predesigned programs.

In one embodiment, the user interface module may include one or more of knobs, switches, keys, keypad, indicator light, LCD or other means of display, and/or speakers to collect user's inputs or provide feedback to user.

In one embodiment, the device could be made small to be portable or wearable like a watch, or large enough with multiple pairs of electrodes to treat multiple subjects at the same time in a clinic or hospital setting. In another embodiment, it could also be incorporated in other equipment, system or structure to supply treatment/cleansing waveform to the whole building, or other blood/body fluid storage facility. In another embodiment, it can also be merged with other technology such as watch, blood pressure monitor, or other equipment to provide therapeutic or cleansing function.

In one embodiment, when the device is used to cleanse a volume of body fluid (e.g. blood), the electrodes may be of certain shapes to facilitate power transferring to the fluid. In another embodiment, it can be simply connected to electrodes in the vessel/container containing the fluid if the vessel/container itself contains certain built-in electrodes to facilitate power transferring.

In one embodiment, the device would be battery powered, small and wearable like a watch. In one embodiment, it would have only one fixed frequency or one program for any pathogen related ailments. User would wear it and attach the two electrodes to the two points where Radial artery and Ulnar artery pass beneath the skin at the wrist where the device is worn. In one embodiment, the user will then select the length of treatment from a few options: for example, 30 minutes, 60 minutes, 120 minutes, 240 minutes. Then turn a knob to switch on the output and select the output voltage which should be as strong as possible yet does not cause any uncomfortable feeling at the points where the electrodes contact the skin. The treatment then starts and the device will terminate at the selected length of treatment with a signal such as sound. In one embodiment, this would be of great convenience for subjects having infectious disease such as flu. Anywhere anytime the treatment can be started and effect will be seen in 30 minutes or 1 hour, as revealed in the examples below. In another embodiment, since the device is small and wearable, the wearing does not cause any uncomfortable feeling; people can continue their daily activities such as office work, household work, or even exercise as normal.

In one embodiment, the present invention provides many benefits, since the same device can be used for kids, adults or seniors to treat any infection in the blood, without the need for detailed blood test and diagnosis.

In one embodiment, the present invention is useful for disease prevention. For example, this is done by wearing the device 30 minutes after suspected exposure to pathogen. Subjects of some infectious disease might have no symptom initially. For example, when one attends a crowded social event during flu season. It may not be rational to visit a doctor before flu symptoms begin. But when the symptoms are observed, it may be too late to stop the flu. However, one could begin to wear the device after the social event to treat for 30 minutes to prevent the potential flu. Family members can be so protected when kids catch flu from schools. Kids can wear the device for 30 minutes a day when there are other kids having flu. Seniors can avoid many tragedy events by having such a device and wearing it after suspected pathogen exposure.

In one embodiment, the present invention disclosed that lower frequency produces better therapeutic effect. Very low frequency from 3 Hz to 30 Hz will affect the blood cell permeability. In one embodiment, the ideal frequency could be picked between 30 Hz to 100 Hz.

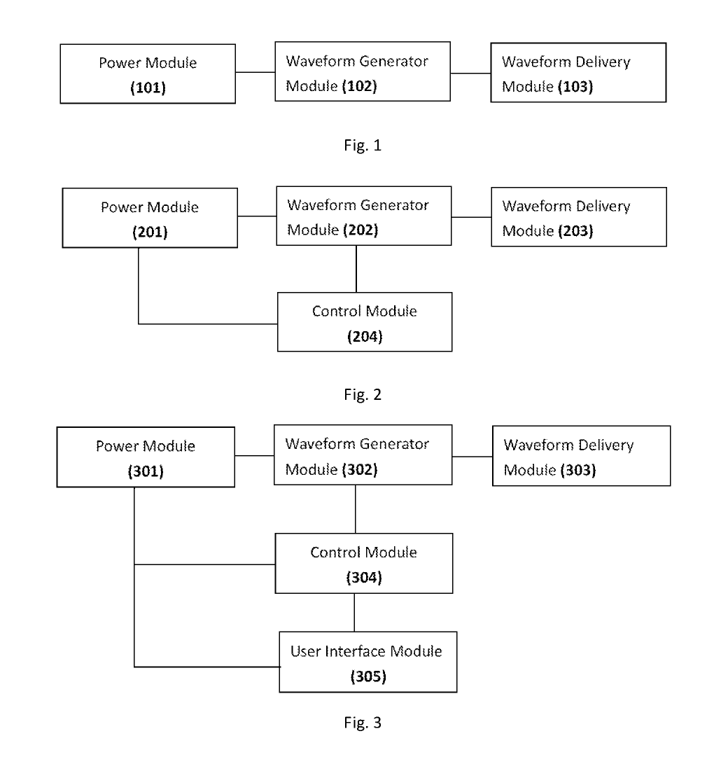

In one embodiment, as shown in FIG. 1, the present invention provides a microbial cleaning system that may include a power supply module 101, a pulse generator module 102 and a pulse output module 103. The power supply module 101 is connected with the pulse generator module 102, the pulse generator module 102 is connected with the pulse output module 103, the pulse output module 103 comprises at least two electrodes connecting the blood or other liquid to be cleaned, and the blood or other liquid to be cleaned has the microorganism to be cleaned;

In one embodiment, the pulse output module 103 is used to apply the pulse signal to the blood or other liquid to be cleaned, so as to clean the mentioned microorganism.

In one embodiment of the microorganism cleaning system, the system is used to clean microorganisms in blood or other liquid. The system includes a power supply module 101, a pulse generator module 102 and a pulse output module 103. The power supply module 101 is connected with the pulse generator module 102, and module 101 is used to supply power for the pulse generator module 102. The power supply module 101 can include either a DC or an AC power source: for DC power supply, it is recommended to use a storage battery whose power supply voltage is matched with the operating voltage of the device; for DC or AC power supply with voltage different from the operating voltage, a voltage converter can be set at the output of the DC or AC power supply, so as to convert the output voltage into an operating voltage suitable for the pulse generator module 102.

In one embodiment, the described pulse generator module 102 is used to generate pulse signals for cleaning microorganisms, and the frequency range can be 4 Hz-10 kHz. The pulse generator module mainly comprises a pulse signal generating circuit. The specific circuit structure of the pulse signal generating circuit can be various, mainly comprising an electromagnetic signal generator, a pulse frequency control unit, a pulse intensity control unit, etc. Pulse signal generating circuits capable of generating a pulse signal with excitation frequency, such as digital circuit, analog circuit, or analog+digital circuit, are all applicable to the present embodiment.

In one embodiment, the input end of the pulse output module 103 is connected with the output end of the pulse generator module 102; the output end of the pulse output module 103 comprises at least two electrodes, and the two or more electrodes communicate with each other by blood or other liquid to be cleaned, forming a closed loop circuit with the pulse generator module 102. The electrodes and the blood or other liquid to be cleaned can be directly or indirectly connected, that is, the connection can be realized either by directly inserting the conductive needles into the blood or other liquid to be cleaned, or by skin and tissue conduction with the blood or tissue fluid. The pulse output module 103 is used to apply the pulse signal to blood or other liquid to be cleaned.

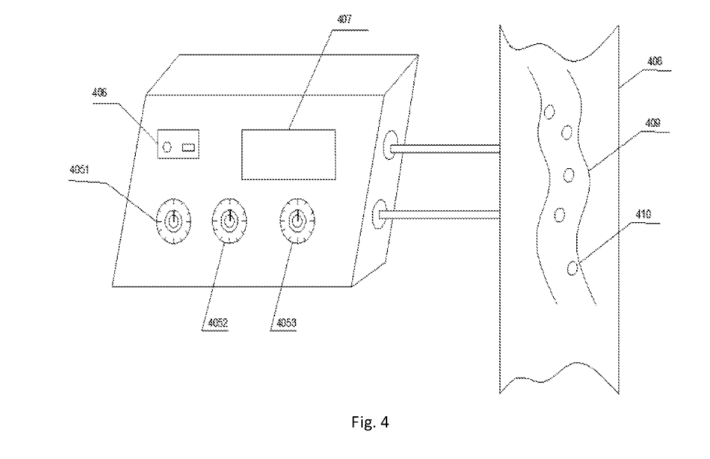

In one embodiment, a switch 406 (FIG. 4) can also be arranged after the power supply module 101. The switch is used to manually control the conduction and non-conduction of the subsequent pulse generating circuit after the power supply module 101 is connected to the power supply, so as to ensure the safety of the user and make it convenient for the user to control and use.

In one embodiment, before the described microorganism cleaning device starts the cleaning operation, connect the two or more electrodes of the pulse output device to various treatment positions of blood or other liquid to be cleaned that contains microorganisms to be cleaned, then turn on the switch 406 to activate the pulse generator module 102, so the described pulse generation circuit will form a complete closed loop with the blood or other liquid to be cleaned. Once the pulse signal generating circuit of the pulse generator module 102 generates a pulse signal with excitation frequency, the pulse output module 103 will output the pulse signal to the conductive blood or other liquid to be cleaned.

In one embodiment, as shown in FIG. 2, another microbial cleaning device is provided, which, on the basis of the above mentioned embodiment, includes an additional control module 204. The control module 204 is connected to both the power supply module 201 and the pulse generator module 202.

In one embodiment, the control module 204 is configured to generate a control instruction to the pulse generator module 202 based on the preset control information. The control information includes pulse interval, frequency, waveform, voltage, etc. The control instructions include pulse generation intervals and pulse parameter control instructions such as frequency, waveform and voltage.

In one embodiment, the pulse generator module 202 is configured to generate a pulse signal of the pulse parameters according to the obtained pulse generation interval and pulse parameter control instruction.

In one embodiment, the present invention provides a micro-organism cleaning device that has an additional control module 204, which is connected to the power supply module 201 and the pulse generator module 202 to control the interval and related parameters of the output pulse signal generated by the pulse generator module 202. The control module 204 has preset control information, and such control information may include pulse generation interval used for controlling the generation interval of pulse signals. Moreover, the control information can also include frequency information, waveform information etc. The control module 204 generates pulse parameter control instruction based on the preset pulse parameter control information. The generated instruction is then transmitted to the pulse signal generating circuit of the pulse generator module 202 at the pulse generation interval.

In one embodiment, when the pulse generator module 202 receives a pulse parameter control instruction from the control module 204, the pulse generation circuit generates electronic pulses according to the pulse parameters control instruction received, and this cycle repeats.

In one embodiment, when user connects the pulse output module 203 of the microbial cleaning device to treatment positions of blood or other liquid to be cleaned. Turn on the switch to actuate the control module 204 and the pulse generator module 202. The control module 204 will generate control instructions based on its internally preset control information (for example, a pulse generation interval of 300 ms, and pulse signal parameters as: a pulse signal frequency of 20 Hz, a pulse signal current of 4 mA). The control instruction is generated at a time interval of 300 ms, the control instruction includes pulse parameter control instructions (the generation of pulse signal with the following parameters: pulse signal frequency: 20 Hz; pulse signal current: 4 mA). The pulse generator module 202 receives the pulse generation parameter control instruction at an interval of 300 milliseconds; each time the pulse signal generating circuit receives a pulse parameter control instruction, it generates the preset parameter pulse signal (pulse signal frequency: 20 HZ; pulse signal current: 4 mA).

In one embodiment, as shown in FIG. 3, the present invention provides another microorganism cleaning device. The user interface module 305 is also included on the basis of the above-described embodiments, and this module is connected to both the power supply module 301 and the control module 304.

In one embodiment, the user interface module 305 is configured to receive control information input from the user, and to present work information to the user. The control information includes voltage, treatment time, waveform, etc. The information to be presented to the user also includes battery capacity, control information of the ongoing treatment, and sound prompt for treatment completion. The prompt information is provided via audio or visual signals by means of relevant components, such as a loudspeaker, an indicator lamp or a LED display screen.

In one embodiment, the control module 304 is specifically configured to generate pulse parameter control instructions to the pulse generator module 302, based on the user control information it obtained from user interface module 305, so that the pulse generator module 302 can generate pulse signals according to the user control information.

In one embodiment, the microbial cleaning device provided by the present invention has an additional user interface module 305, which is powered by the power supply module 301. The output of the user interface module 305 is connected to the control module 304, so the user-input control information received by module 305 can be transmitted to the control module 304, and such information includes pulse parameter information such as the waveform, voltage, treatment duration and other parameters. The user interface module can receive user-input control information in various ways. For example, users can manually input the information, or convert the control information into data format and transmit the data to the user interface module 305 through a cable or wireless connection. The control module 304 generates a pulse parameter control instruction to the pulse generator module 302 based on the control information it obtained, and the pulse parameters control instructions can include pulse frequency control instruction, pulse waveform control instruction, etc. The pulse signal is then generated by the pulse generating circuit of the pulse generator module 302, and then input to blood or other liquid to be cleaned by the pulse output module 303 to clean up the microorganisms in blood or other liquid to be cleaned.

In one embodiment, in the microbial cleaning device provided by the present invention, a user interface module is added to conveniently receive the control information input by the user.

In one embodiment, the present invention provides another microbial cleaning device in which, on the basis of the above-mentioned embodiments, the pulse generator module includes a first output terminal and a second output terminal, and the pulse output module includes a first output electrode and a second output electrode. The first output terminal of the pulse generator module is connected to the fixed terminal of the first output electrode; the second output terminal is connected to the fixed terminal of the second output electrode; the free end of the first output electrode and the free end of the second output electrode are in communication through blood or other liquid to be cleaned.

In one embodiment, in the microorganism cleaning device, the pulse output module that outputs pulse signals is comprised of a first output electrode and a second output electrode; the first output electrode and a second output electrode are respectively connected to the first output terminal and the second output terminal of the pulse generator module to input the pulse signal of the pulse generating circuit to blood or other liquid to be cleaned, so that the microorganisms in blood or other liquid to be cleaned can be cleaned up.

In one embodiment, on the basis of above-described embodiments, a safeguard conductive structure can also be set up. Both the free end of the first output electrode and the free end of the second output electrode are provided with a safeguard conductive structure; the free end of the first output electrode and the free end of the second output electrode are in communication with the conductive blood or other liquid to be cleaned through the safeguard conductive structure.

In one embodiment, the microbial cleaning device is mainly used for cleaning up micro-organisms in blood or other liquid to be cleaned, so it is necessary to set up safe conductive structures, which can not only facilitate the conductive connection between pulse signals (which are output by the device) and blood or other liquid to be cleaned, but also guarantee the safety of living bodies.

In one embodiment, on the basis of the above embodiments, the safeguard conductive structure can include self-adhesive medical conductive patches. The safeguard structure can be various forms, for example, a cotton sheath can be placed on the free end of the first output electrode, once the cotton sheath is immersed in dilute sea salt water or saline, it is connected with the skin surface of the blood. Although the above-mentioned safeguard construction is simple in structure, low in cost, and safe in electricity conduction, it is inconvenient to use. Therefore, the micro-organism cleaning device in the present embodiment uses self-adhesive medical conductive patches as its preferred safeguard conductive structure, which can be directly adhered to different positions of the skin required by the pulse signal, achieve the purpose of conducting electricity, and ensure safety.

In one embodiment, in the microorganism cleaning device provided by the present invention, the pulse output module connects the output ends of the pulse generator module to different positions of blood or other liquid through the first output electrode and the second output electrode, and safeguard output structure is arranged at each output end of the electrode. Self-adhesive medical conductive patch is preferred, since it allows cleaning up of the micro-organisms in blood or other liquid to be cleaned with pulse, guarantees convenient usage, and ensures safety.

In one embodiment, the present invention provides another microbial cleaning device, in which the pulse generator module comprises a first output terminal and a second output terminal; the pulse output module comprises multiple output electrode pairs connected in parallel; each of the said output electrode pairs include a positive output electrode and a negative output electrode; For the output electrode pairs, the fixed ends of the positive output electrodes are all connected to the first output terminals, the fixed ends of the negative output electrodes are all connected to the second output terminal, and the free ends of the positive output electrodes are communicated with the free ends of the negative output electrodes through the conductive blood or other liquid to be cleaned.

In one embodiment, the present invention also provides another microbial cleaning device, in which the pulse generator module comprises a first output terminal and a second output terminal; the pulse output module comprises multiple output electrode pairs connected in parallel; each of the said output electrode pairs include a positive output electrode and a negative output electrode; For the output electrode pairs, the fixed ends of the positive output electrodes are all connected to the first output terminals, the fixed ends of the negative output electrodes are all connected to the second output terminal, and the free ends of the positive output electrodes are communicated with the free ends of the negative output electrodes through the conductive blood or other liquid to be cleaned. The electrodes of the multiple pairs of output pulse signals can be connected in a variety of ways. For example, the multiple pairs of electrodes can be applied to different parts of the same human body to form multiple sets of circuits: e.g., apply two pairs of electrodes to both hands or both feet, so the micro-organisms in multiple parts of the blood vessel in the same body can be cleaned up. Meanwhile, it is also possible to apply the multiple pairs of said electrodes to different human bodies, so the microorganisms in the blood or other liquid to be cleaned of multiple human bodies can be simultaneously cleaned up. Such devices are preferably provided in larger devices, such as large equipment in hospitals, which can simultaneously clean up microbes in multiple human bodies, reduce treatment costs, and enhance treatment efficiency.

In one embodiment, as shown in FIG. 4, on the basis of the above mentioned embodiments, the present invention also provides another microbial cleaning device, wherein the user interface module comprises an adjustable knob. The adjustable knob is used to receive the pulse control information input by the user.

In one embodiment, in the microbial cleaning device provided by the present invention, the user interface module for receiving control information input by the user can be implemented in various modes, such as setting the display screen 407, manual input device, a data cable port to import external data, wireless data transmission, and the preferred adjustable knob. The adjustable knob can be set as a manual adjustable knob, with data selection parameters surrounding the knob. The adjustable knob is configured with selection marks, so information input can be realized by turning the selection knob to the desired pulse signal parameter position, and the pulse generator module can then generate pulse signals with the selected parameters.

In one embodiment, please refer to FIG. 4, on the basis of the above mentioned embodiments, the adjustable knobs can include a duration adjustment knob 4051 and a current adjustment knob 4052. The duration adjustment knob 4051 is used to receive the treatment time information input by the user, and the current adjustment knob 4052 is used to receive the current information of the pulse input by the user.

In one embodiment, the duration adjustment knob 4051 is configured with a duration selecting mark, and the duration adjustment data are set in the periphery of the duration adjustment knob 4051. The duration range available for selection is from 5 minutes to 10 hours.

In one embodiment, the current adjustment knob 4052 is configured with a current selection mark, and the current adjustment data are set in the periphery of the current adjustment knob 4052. The current range available for selection is from 0 to 5 mA, because when the current of the pulse signal is in the range of 0-5 mA, the current entering blood vessel through skin is in the range of 50 to 100 μA, so that the current is sufficient to cause damage to the microorganism 410 in the blood 409, but still low enough to ensure that the microbial cleaning device acting on the skin surface of the living body will not cause damage to the biological cell.

In one embodiment, please refer to FIG. 4, based on the above mentioned embodiments, the adjustable knobs can also include a waveform adjustment knob 4053, which is used to receive the waveform information of the pulse in demand input by the user.

In one embodiment, in the microbial cleaning device provided by the present invention, a waveform adjustment knob is added to adjust the output waveform of pulse signals. The waveform adjustment knob is configured with a waveform selection mark, and the waveform selection scope may include: sine wave, square wave, triangular wave, sawtooth waveforms and other waveforms.

In one embodiment, the microbial cleaning device provided by the present invention includes a power supply module that supplies power, a user interface module that receives user input control information, a control module that controls pulse signal parameters, a pulse generator module that generates pulse signals, and a pulse output module that outputs pulse signals to the treatment spot of blood or other liquid to be cleaned; the user can select relevant parameter information through corresponding adjustment knobs.

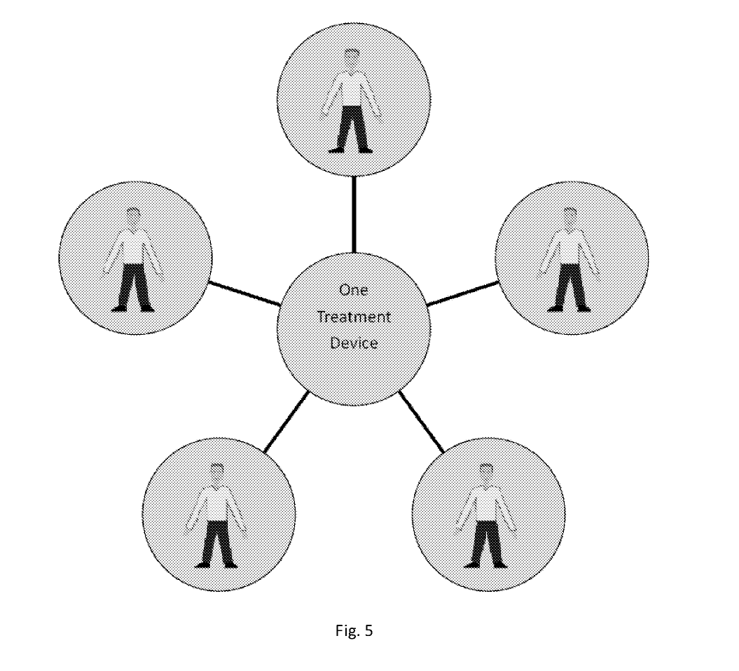

In one embodiment, as shown in FIG. 5, multiple pairs of electrodes can be connected to one treatment device, simultaneously providing treatments to multiple subjects.

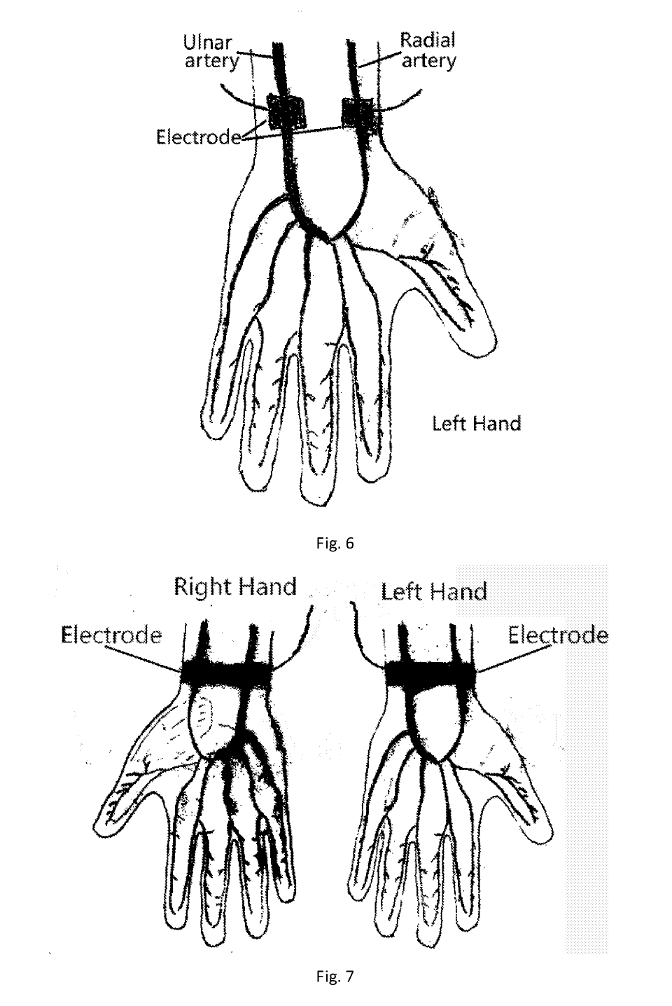

In one embodiment, the electrode pair can be configured by implementing both electrodes on the same body part. As shown in FIG. 6, both electrodes are configured on the same waist. One electrode is close to the ulnar artery and the other is close to the radial artery.

In one embodiment, the electrode pair can be configured by implementing both electrodes on different body parts. As shown in FIG. 7, each of electrodes is configured on different waists, respectively.

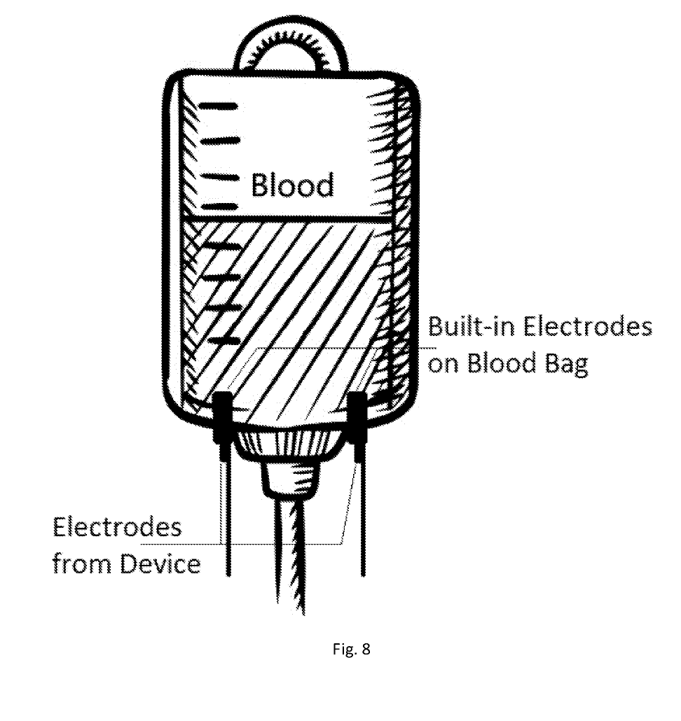

In one embodiment, the electrode pair can be configured by implementing both electrodes on a vessel containing body or synthetic fluid. As shown in FIG. 8, both electrodes are configured on a vessel.

The present invention disclosed a system and method for treating pathogens and related diseases in a subject or disabling pathogens in body fluid or synthetic fluid, comprising at least one pair of conductive electrodes, each of which connects to physically separated points on said subject, or physically separated points on a vessel containing said body fluid or synthetic fluid; and at least one electrical signal generator circuitry which generates oscillatory electrical signal comprising selected single frequency, waveform, electricity, and voltage.

In one embodiment, the invention is characterized by that the frequency is in a range of 4-10000 Hz, that the frequency is fixed for any pathogen or disease, and that no frequency scanning is required to get such value.

In one embodiment, the system is further comprised at least one controlling circuitry which controls the voltage, waveform, electricity and a treatment period.

In one embodiment, the system is further comprised a controlling circuitry together with a user interface module which interfaces between an user and said electrical signal generator to control the voltage, waveform, electricity and a treatment period.

In one embodiment, the invention is characterized by pathogen which is selected from the group consisting of bacteria, virus, fungus, and parasites.

In one embodiment, the invention is characterized the frequency which is in a range of 30-1000 Hz.

In one embodiment, the invention is characterized by the voltage which is adjustable and has an absolute voltage difference between electrodes less than 27 V.

In one embodiment, the invention is characterized by the electrical signal which has an intensity of less than 5 mA and can be adjusted by user to prevent tingling feeling or pain at skin.

In one embodiment, the invention is characterized by the waveform which is bi-phasic or square waveform.

In one embodiment, the invention is characterized by the treatment period which is between 5 minutes and 10 hours per treatment session.

In one embodiment, the invention is characterized the treatment period which is between 15 minutes and 2 hours per treatment session.

The present invention also disclosed a method of treating pathogen or related diseases in a subject in need of such treatment, comprising administering an electrical field to said subject through the system.

In one embodiment, the method is characterized by the diseases which are caused by bacteria, virus, fungus, or parasites.

In one embodiment, the method is characterized by the electrical field which comprises an electricity of less than 5 mA and can be adjusted by user to prevent tingling feeling or pain at skin.

In one embodiment, the method is characterized by the electrical field which is administered in a period ranging from 5 minutes to 10 hours per treatment session.

In one embodiment, the method is characterized by the electrical field which is administered by a subject in a period ranging from 15 minutes to 2 hours per treatment session.

In one embodiment, the method is characterized by the subject which is a mammal.

In one embodiment, the method is characterized by the subject which is a human.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Throughout this application, various references or publications are cited. Disclosures of these references or publications in their entireties are hereby incorporated by reference into this application in order to more fully describe the state of the art to which this invention pertains.

Experiment

A device of a fixed frequency of 39 Hz with a square wave is configured on both waists and utilized for the following experiments unless explicitly described otherwise. A voltage between 7V to 18V, which is the maximum effective voltage without causing tingling feeling or pain at the electrode contact points, is selected by the user for the treatment unless explicitly described otherwise.

Difference Effect in Comparison with Hulda Clark's Device

A family including a male at age of 39, a female at age of 35, and two girls at ages of 2 and 5, respectively. The family used the Hulda Clark device of 30 kHz for about a year. It was noticed by the family that the flu symptoms during a flu season including fever, sore throat, muscle pain, cough etc. became less severe. Especially when such device was used on the two girls, high fever became mild fever. The flu symptoms stop in 3-5 days after use of the Hulda Clark device, indicating that the fixed frequency device is useful to make the flu mild. However, it could not completely stop the flu in one or two treatments. Later, the family began to use the devices of the present invention with a fixed frequency of 1 kHz, 100 Hz, 40 Hz, and eventually 4 Hz. It was noticed that a lower frequency provides faster resolution of the flu. With a treatment with a fixed frequency of 4 Hz, the flu would be completely cured after 2-3 days. When the voltage was increased from 6V to 15V, the flu can be completely resolved within 1-2 days. As for the treatments implemented on the two little girls, the flu got healed in one treatment of 1-2 hours.

Use of Device to Treat Flu

A female patient at age of 37, symptoms of flu: sore throat, muscle aches, sneezing, serious nasal congestion, constant tearing, runny nose. Because of the serious symptoms, the patient had to use paper wipe consistently and her work has to be stopped. After utilizing the device for 2 hours starting around noon, the symptoms such as nasal congestion and runny nose were significantly alleviated, and the patient could resume working without using any paper wipe. After another utilization of the device for overnight, the patient felt much better and continued to work as usual. She thought she already was fully recovered and stopped using the device. The symptoms such as nasal congestion and runny nose came back in the afternoon. After using the device for 2 hours, the nasal congestion and tearing symptoms were alleviated a lot. The patient continued to use the device one time per day for another two days and was fully recovered. The patient didn't take any drug during the period. The patient usually used to suffer from cough with sputum, purulent nasal discharge, or inflammation of sinus for couple weeks after a flu. This time these symptoms were not observed after the treatment by the device disclosed in the present invention.

A female infant at age of 1 and her mother at age of 30, both suffering from flu with typical flu symptoms. On the third day of flu, the infant was described to be crying all night. Both the baby and the mother volunteered to use the device, setting the electrical field at small strength of 9V. Both patients felt much better after first use of 30 minutes. Both took another treatment for another 2 hours. The infant was fully recovered on the next day. The mother continuous to use the device at minimum voltage of 9V for another five or six days until fully healed.

A girl at age of 7 and a boy at age of 8 in the same neighborhood took the same bus to the same school. The boy has taken one day off for flu symptoms. The second morning the girl got same symptoms. Immediately the parents of the girl used the device on the girl for two 1-hour treatments in the morning and afternoon, respectively. The girl got fully recovered and went back to school on the next day. Her families were not affected. The boy, without using the device, was fully recovered after more than one week and his father also caught the severe flu.

A male at age of 55 with typical symptom of flu such as runny nose and a tiring feeling. He took the device and used the maximum voltage of 18V in order to get well fast. He fully recovered after two days. He didn't feel anything uncomfortable at the wrists where the electrodes were attached.

A female at age of 50 with typical symptom of flu such as sore throat, point pain, runny nose, headache, and a feeling of vomit. After using the device, she felt much better and was fully recovered after several days.

A male at age of 42. He used the device to treat flu for a couple of times, in which the symptoms can be significantly alleviated after wearing the device for 1 hour. The symptoms were completely gone after a 2 hour-wearing, or no more than two 2-hour wearing. The treatment also did not lead to sore throat, runny nose, fever, aches or other symptoms.

Use of Device to Treat Unknown Infection

A female computer engineer at age of 39, on a sick leave for 3 months. She had a pale face and were feeling always tired. She was suggested by a physician to take some anti-depression drugs. She indicated that she had flu symptoms for the whole period of 3 months. The symptoms were not alleviated even after taking various antibiotics. She volunteered to use the device. After three days she was fully recovered and the flu symptoms disappeared.

A girl, 3 years old, had a weird symptom of not eating much, not having regular bowel movement, low energy, and recurring flu symptoms in about every month. The mother volunteered to put the device on the girl when the flu symptoms happened again. The symptoms stopped in an hour of using the device. But the second day, similar symptoms came back. After using the device for one hour, all symptoms stopped, except a bad running nose happened. The third day and fourth day, the mother continued to use the device on the girl everyday one hour, no other symptoms except the running nose. At the fifth day, the running nose stopped. The girl began to have much better appetite and regular bowl movement, much better energy. And the flu symptoms did not come back the next month.

Use of Device to Treat Cold

A female teenager at age of 13 got cold after attending a wedding. She suffered from muscle aches and were feeling tired with symptoms of cold since the night after the wedding. The symptoms became worse on the second day and she had a fever of 40° C. at night. That night after using the device for 1 hour, the teenager felt much better. The teenager continuously worn the device overnight and got fully healed on the following day. No cough or purulent nasal discharge was observed. Wearing the device did not bring any uncomfortable feeling.

A girl at age of 7 got cold during night due to low temperature and not covered by blanket while sleeping. In the next morning, she suffered from sneezing and runny nose. The girl immediately worn the device; the symptoms disappeared after one hour treatment; and the girl stopped wearing the device after two hours and was fully recovered.

Use of Device to Treat Fever

A female at age of 33 was feeling tired and suffered from headache and muscle aches for two days. In the late afternoon, she got a fever of 38° C. She started to wear the device. After two hours, she woke up naturally with a sweaty body and a normal body temperature. All symptoms such as headache and muscle aches disappeared. No sneezing or runny nose was ob served.

Use of Device to Treat Common Cold (Small Clinical Trial)

This is a small single-arm clinical trial on eleven kids ranging from 3 years old to 46 years old. They were diagnosed as having common cold, most with a fever. They all filled the consent form to use the device in addition to standard treatment prescribed by the doctor. They were required to wear the device on left/right wrists for 3 hours/session. And they can use it multiple times in two days according to their cold symptom. Outcome of the cases are summarized below:

| Category | Treatment and Outcome | Number of Patients | Percentage |
|---|---|---|---|
| 1 | Cured right after | 5 | 38.4% |
| | one treatment | | |
| 2 | Cured after multiple | 6 | 46.2% |
| | treatment in 2 days | | |
| 3 | Not cured in multiple | 2 | 15.4% |
| | treatments in 2 days | | |

There were 4 patients did not finish even one treatment of 3 hours. 3 of them were treated 1 hour and 1 of them was treated 2 hours. They then discontinued the treatment and left the hospital with medicines prescribed by the doctor. Their leave was not because of any side effects, and was because they did not expect this treatment would help them. Since they did not finish even one required treatment, they were not included in the above analysis.

The device did show its effect for patients in category 1 and 2, particularly the 5 patients that were cured after one treatment. That was fast cure for colds that needed help from hospital. We confirmed the data by calling each patient once per day in a two day period after they left the hospital.

1. A system for treating a disease or improving the well-being of a subject by applying an oscillatory electrical signal for an effective amount of treating time, said system comprising:
at least one pair of conductive electrodes, each of which is configured to contact physically separated points on said subject; and
at least one electrical signal generator circuitry that generates said oscillatory electrical signal;
wherein said electrical signal has a fixed frequency selected from the range of 4-10000 Hz,
wherein voltage difference between said electrodes is in the range of 7-18 V.

2. The system of claim 1, further comprising at least one controlling circuitry that controls the treating time, and voltage or waveform of said electrical signal.

3. The system of claim 1, further comprising a user interface module for adjusting the treating time, and voltage or waveform of said electrical signal.

4. The system of claim 1, wherein said electrical signal has a fixed frequency selected from the range of 30-1000 Hz.

5. A method of treating a disease or improving the well-being of a subject by using a system, said system comprising:
at least one pair of conductive electrodes, each of which is configured to contact physically separated points on said subject; and
at least one electrical signal generator circuitry that generates said oscillatory electrical signal; said electrical signal has a fixed frequency selected from the range of 4-10000 Hz, voltage difference between said electrodes is in the range of 7-18 V;
said method comprising:
i. configuring the conductive electrodes of said system to contact physically separated points on said subject;
ii. selecting a voltage and waveform for an electrical signal; and
iii. applying said electrical signal to said subject for an effective amount of treating time.

6. The method of claim 5, wherein the waveform is a bi-phasic square waveform.

7. The method of claim 5, wherein the treating time is between 5 minutes and 10 hours per treatment session.

8. The method of claim 5, wherein said electrical signal has a fixed frequency selected from the range of 30-1000 Hz.

9. The method of claim 5, wherein said disease is caused by bacteria, virus, fungus, or parasites.

10. The method of claim 5, wherein said subject is less than 18 years old.

11. The method of claim 5, wherein said subject is a medicine-taker or alcohol drinker.

12-19. (canceled)

20. The system of claim 1, wherein said disease is flu, cold, or fever.

21. The system of claim 4, wherein said electrical signal has a fixed frequency selected from the range of 30-100 Hz.

22. The system of claim 1, wherein said disease is caused by bacteria, virus, fungus, or parasites.

23. The system of claim 1, wherein said subject is less than 18 years old.

24. The system of claim 1, wherein said subject is a medicine-taker or alcohol drinker.

25. The method of claim 5, said system further comprising at least one controlling circuitry that controls the treating time, and voltage or waveform of said electrical signal.

26. The method of claim 5, said system further comprising a user interface module for adjusting the treating time, and voltage or waveform of said electrical signal.

27. The method of claim 8, wherein said electrical signal has a fixed frequency selected from the range of 30-100 Hz.

28. The method of claim 5, wherein said disease is flu, cold, or fever.
